(19) United States
(12) Patent Application Publication
(10) Pub. No.: US 2023/0355972 A1
(43) Pub. Date: Nov. 9, 2023

(54) NEUROMODULATION FOR THE TREATMENT OF CRITICAL ILLNESS

(71) Applicant: Queen Mary University of London, London (GB)

(72) Inventors: Gareth Lewis Ackland, London (GB); Andrey Gourine, Stockholm (SE)

(21) Appl. No.: 18/017,500

(22) PCT Filed: Jul. 23, 2021
(86) PCT No.: PCT/EP2021/070749

§ 371 Date: Jan. 23, 2023

(30) Foreign Application Priority Data
Jul. 24, 2020 (GB) 2011523.4

(51) Int. Cl. A61N 1/36

(52) U.S. Cl. CPC A61N 1/36036; A61N 1/36034

(57) ABSTRACT

There is provided a method for the treatment of a patient suffering or recovering from a critical illness using a device comprising a generator configured to produce an electrical stimulation signal and a controller. The controller is connected to the generator and configured to determine the form of the electrical stimulation signal. The method includes producing an electrical stimulation signal from the generator, and determining the form of the electrical stimulation signal using the controller connected to the generator; and transmitting the electrical stimulation signal to an electrode in contact with a tragus of the patient.

### RELATED APPLICATIONS

This application claims priority from Application PCT/EP2021/070749, filed Jul. 23, 2021, and claims priority from Great Britain Patent Application No. 2011523.4, filed Jul. 24, 2020, each of which are incorporated by reference in their entireties in this application.

### FIELD OF THE INVENTION

The present invention relates to a method of non-invasive neuromodulation by application of a specific programme of electrical stimulation signals to cutaneous sensory projections of cranial nerves for use in the treatment of critical illness and rehabilitation after critical illness in human subjects.

### BACKGROUND OF THE INVENTION

An essential part of any modern hospital treatment facility is an intensive care unit (ICU) (also referred to as intensive therapy unit (ITU)) which is designed for the recovery of patients with critical illnesses requiring significant and/or frequent intervention. Any patient that needs treatment in an ICU can often also suffer from additional complications including ventilator dependency, muscle wasting, increased weakness and/or impaired longer term physical and neurocognitive function.

A cardinal feature of critical illness is the emergence of autonomic dysfunction (also termed “dysautonomia”), characterised by sympathetic hyperactivity and loss of parasympathetic (vagal) function. Persistent autonomic dysfunction directly impairs cardiorespiratory fitness and therefore muscle mass and strength, key determinants of functional recovery, cannot be increased. Furthermore, disruption of the immunoregulatory role of the autonomic nervous system establishes a pro-inflammatory state characterised by suppression of innate mechanisms that inhibit inflammation. Autonomic dysfunction combined with persistent inflammation prevents functional recovery and accelerates post-discharge cardiovascular and infectious morbidity.

It has been shown that autonomic dysfunction develops rapidly after major surgery that requires intensive care and is associated with more complications.

In the United Kingdom, post-ITU and hospital discharge, patient rehabilitation is typically provided by the responsible hospital. In the UK, estimates put the number of ITU critical care patients at 140,000 per year with around 70% survival rate. The typical cost for rehabilitation per patient is ˜£50 k (£7 k-£250 k range). Rehabilitation in this context may mean functional rehabilitation, defined as a return to premorbid levels of function and/or activity.

There is therefore a need for rehabilitation of patients suffering from a critical illness, particularly an illness such as the COVID-19 infection as these patients appear to require a significant recovery period.

Despite significant advances in medical research, the biological/physiological mechanisms that underlie the recovery from, and/or determine the speed of recovery from, critical illness are not fully understood.

Devices for stimulation of the cranial nerves are known from WO 2018/050773.

### SUMMARY OF THE INVENTION

The present invention relates to the realisation that therapies that augment efferent parasympathetic (vagal) activity and/or reduce excessive sympathetic drive should restore autonomic balance, enhance adrenoreceptor sensitivity, lower heart rate and restrain inflammation in a patient suffering from autonomic dysfunction. Hence, redressing of the autonomic nervous system balance can minimise the adverse consequences of autonomic dysfunction triggered by acute injury/critical illness.

In particular, the present invention relates to the discovery that use of non-invasive autonomic neuromodulation to aid recovery from critical illness may offer an improved path to facilitated functional recovery and wellness for patients. More specifically, the present disclosure is related to the field of critical illness treatment of medical conditions leading to failure of one or more organ(s). Said conditions include, but are not limited to, viral, bacterial and/or fungal infection (e.g. coronavirus infection and disease that arises therefore, for example, COVID-19, or sepsis), post-surgical recovery, post-traumatic stress disorder (PTSD), sleep apnoea, organ failure, e.g. failure of the heart, lung, brain, kidneys and/or other major organs (where the organ failure may be due to injury or disease), cancer, heart attack, organ transplant (e.g. heart, liver, kidney, lung transplant), coronary bypass surgery and angioplasty.

The present invention describes a method of non-invasive electrical stimulation of sensory nerves that project to the skin of the outer ear for treatment of patients with a critical illness in order to aid recovery. Recovery in this sense may mean functional recovery defined as progressive return of the patient to a pre-disease (premorbid) level of physical fitness and activity. This can be achieved non-invasively by stimulation of cutaneous sensory projections of several cranial and spinal nerves that originate from the brainstem and spinal cord. Non-invasive treatment includes both transcutaneous or percutaneous contact with the skin. Transcutaneous contact is when the electrical stimulation signal is applied to the surface of the skin, whereas percutaneous contact involves using electrodes to penetrate through the skin and deliver the electrical stimulation signal. Regions of the external ear, and the tragus in particular, are innervated by the sensory branches of the fifth (V) and the tenth (X) cranial nerves as well as branches of spinal nerves C2 and C3. Thus, these nerves can be stimulated according to the methods of the present invention.

In a first aspect of the invention there is provided a method for the treatment of a patient suffering from a critical illness and/or recovering after a critical illness using a device comprising a generator configured to produce an electrical stimulation signal and a controller, connected to the generator and configured to determine the form of the electrical stimulation signal, the method comprising the steps of producing an electrical stimulation signal from the generator; determining the form of the electrical stimulation signal using the controller connected to the generator; and transmitting the electrical stimulation signal to an electrode in contact with the tragus of the patient. The step of producing an electrical stimulation signal from the generator may occur before, simultaneously with or after the step of determining the form of the electrical stimulation signal using the controller connected to the generator.

The electrical stimulation signal may comprise a series of electrical pulses, where each pulse may repeat with a frequency of 1 Hz to 100 Hz and each pulse may have a duration of 10 microseconds to 500 microseconds and an amplitude of 0.1 mA to 8 mA. The device may be applied to the patient at least once a day. The device may be applied to the patient on multiple consecutive days e.g. for up to 2, 3, 4, 5 or more consecutive days, for example, at least 5 consecutive days. In another example, in certain cases, the device may be applied to the patient for 7-10 consecutive days.

The device may also suitably include an earpiece, connected to the generator and controller and wherein the earpiece has an electrode. The controller can transmit the electrical stimulation signal to the electrode and the electrode may be configured to be placed in contact with and provide the electrical stimulation signal to a tragus of the patient.

The critical illness may be a disease or condition selected from the group consisting of COVID-19 infection, post-surgical recovery, trauma, post-traumatic stress disorder (PTSD), sleep apnoea, infection (viral, bacterial and/or fungal infection, including sepsis), organ failure, e.g. failure of the heart, lung, brain, kidneys, bone marrow and/or other major organs, cancer, heart attack, organ transplant (e.g. heart, liver, kidney, lung, bone marrow transplant), coronary bypass surgery and angioplasty. The organ failure may include failure of bone marrow associated with chemotherapy to treat cancer, i.e. side effects of chemotherapy to treat cancer.

The infection may be a viral infection, a bacterial infection, and/or a fungal infection. Patients with a critical illness arising from a viral infection may be particularly suitable for treatment according to a method of the invention. Viral infections and associated disorders suitable for treatment according to a method of the invention, include but are not limited to the treatment of a coronavirus infection, such as for example severe acute respiratory syndrome coronavirus 2 (SARS-CoV-2), responsible for the disease condition referred to as COVID-19 infection.

The device can be applied to the user such that the electrical stimulation signal is applied transcutaneously or percutaneously to the user.

The device can be applied to the user for a minimum of 5 minutes and a maximum of 2 hours per day, for example when using at least once per day. For example, the device is applied to the user for at least 15 minutes.

The device may have a first and second electrode, the first electrode can be configured to be placed in contact with the left tragus of the user and the second electrode can be configured to be placed in contact with the right tragus of the user. Additionally, the device may have a first earpiece and a second earpiece, and the first earpiece may contain the first electrode and the second earpiece may contain the second electrode.

The device may further include a securing means configured to secure the electrode to a tragus of a user. The securing means may include a clip and the clip may include a first gripping portion and a second gripping portion which are biased into contact with each other. The electrode may be located on the first gripping portion.

There may also be a reference electrode located on the second gripping portion. The reference electrode may be any kind of sensor (e.g. temperature, physiological).

The device may include a physiological sensor configured to measure the value of a physiological parameter and store the value in a memory portion of the device. The device may also include a temperature sensor configured to measure the temperature of the skin of the tragus and store the value in a memory portion of the device. The value stored in the memory portion may be used by the controller to determine the form of the electrical stimulation signal. The physiological sensor and/or the temperature sensor may be located on the clip.

The physiological sensor measurements, temperature sensor measurements and time and date information on the use of the device by the patient may be recorded and stored in the memory portion of the device.

Additionally, measurements of current, voltage and phase relationship of the electrical stimulation signal may be stored in a memory portion of the device and used to determine the electrical impedance of the tragus.

The controller may be configured to produce the electrical stimulation signal and the pattern of stimulation based on a user input received at the controller. The user input may include at least one of the pulse duration, waveform, pulse frequency, pulse pattern and current amplitude of the electrical stimulation signal.

The present invention provides a method for a very specific means of autonomic nervous system neuromodulation via stimulation of the autonomic nerve system. This method of stimulation (anatomical positioning of the electrode, stimulating signal, duration of stimulation and number of treatment sessions) has been shown to significantly reduce systemic arterial blood pressure, increase exercise capacity with the effect lasting many weeks/months after treatment has stopped, after which treatment can be repeated.

The invention provides a novel means of supporting recovery from critical illness and/or major surgery.

According to the present invention, autonomic neuromodulation via nerve stimulation is highly effective in reversing autonomic dysfunction, which is critical for improving physical fitness. For patients who have suffered a serious injury or critical illness, post-hospitalisation, conventional rehabilitation programs typically require intensive professional support over prolonged periods and invariably incur high healthcare costs.

The present invention provides a device-based treatment solution which applies a low-voltage electrical stimulation signal to the outer ear using a unique stimulation algorithm. This simple, safe, non-invasive stimulation has been shown to rapidly improve cardiovascular function. This invention potentially provides a low-cost, self-administered solution that can be made rapidly accessible to thousands of hospitalised/affected individuals.

Advantageous features of the invention include:
specific targeting of many hospitalised (but likely underserved) patients with ongoing, subclinical pathology amenable to therapeutic intervention.
therapeutic intervention based on novel patent-protected non-invasive autonomic neuromodulation, using the gold-standard format of a randomised, blinded controlled trial.
systems biology approach to integrate trial intervention with biological understanding of the recovery process.
understanding mechanisms underpinning intermediate and long-term pathological sequalae (complications) of infection (including COVID-19 arising from infection from coronavirus).
highly scalable, cost-effective rehabilitation.
accelerating recovery from serious injury/critical illness for the majority of hospitalised patients will reduce the societal and economic impact and help prevent the development of morbidity/multi-morbidity.

Example features of the second and subsequent aspects of the invention are as for the first aspect mutatis mutandis.

The device may be provided as part of a system for modulating the electrical stimulation signal generated. The system may include the device and further comprise a communication module connected to the controller of the device. The communication module may be configured to send information from the device to an external computer system and to receive information from the external computer system, and the information received from the external computer system is used by the controller to determine the form of the electrical stimulation signal.

The electrode can be configured to be placed in contact with an outward facing surface and an inward facing surface of the tragus. The device may include a pair of electrodes. The pair of electrodes comprises a first electrode in contact with an outward facing surface and an inward facing surface of the tragus. The electrical stimulation signal is applied by the first electrode and penetrated across the tragus to the second electrode. In this way a net current flow is achieved across the tragus.

The electrical stimulation signal may be transmitted to at least a first and second electrode, and the first electrode may be configured to be placed in contact with the left tragus of the user and the second electrode may be configured to be placed in contact with the right tragus of the user. The electrical stimulation signal applied to the left and right tragi may be substantially the same electrical stimulation signal and may be applied simultaneously or sequentially to each of the left and right tragi. Alternatively, the electrical stimulation signal applied to the left tragus may be different to the electrical stimulation signal applied to the right tragus and the electrical stimulation signal applied to the left and right tragi may be applied simultaneously or sequentially to each of the left and right tragi. Alternatively, the electrical stimulation signal applied to the left tragus may be different to the electrical stimulation signal applied to the right tragus and the electrical stimulation signal applied to the left tragus may be applied at a different time to the electrical stimulation signal applied to the right tragus.

When using the device and applying the method of treatment, the electrical stimulation signal can be of a sinusoidal, square, triangular, pulse or “white noise” waveform. The electrical stimulation signal may be a pulse waveform, the pulse being substantially a sinusoidal, square, triangular, or “white noise” waveform. The generated waveform may be a symmetrical monophasic waveform, or a symmetrical biphasic waveform, or a symmetrical triphasic waveform. The generated waveform can be an asymmetrical monophasic waveform, or an asymmetrical biphasic waveform, or an asymmetrical triphasic waveform.

The method of the invention may be applied to the user for a minimum of 5 minutes and a maximum of 2 hours per day, for example, for at least 15 minutes. The device can be applied to the user for a minimum of 5 minutes and a maximum of 2 hours per day, for example, for at least 15 minutes.

The method or device may be applied to the user for a minimum of 5 minutes and a maximum of 2 hours per day, for example, for at least 15 minutes, and the method can be applied at intervals separated by at least one day.

The method or device can be applied to the user using different periods. For a first period, the method may be applied to the user for between 5 minutes and 2 hours each day (for example, at least 15 minutes), the first period comprising a minimum of 3 consecutive days. During a second period the method may be stopped for at least 2 days. During a third period the method may be applied to the user for between 5 minutes and 2 hours each day (for example, at least 15 minutes).

The method or device may be applied to the user in combination with any medication(s) administered according to guidelines for modulation of pharmacological effect. The specific medication(s) may be determined based on the specific critical illness, symptoms and/or recovery plan for a specific user, as determined by the clinician.

The methods of treatment of the invention may therefore further optionally comprise a step of administering to the patient a pharmaceutically active composition for treatment of a disease or condition relating to a critical illness.

The present inventors have also identified that a certain sub-population of critical illness patients are particularly suitable for treatment according to a method of the invention as described herein. These patients are expected to respond particularly well to said treatment. Thus, the present invention also provides a method of screening a patient suffering or recovering from a critical illness for treatment according to a method of the invention as described herein, the method comprising recording an electrocardiogram of a patient for a minimum period of 1 minute; analysing the power spectrum of heart rate variability; determining the low frequency (LF) to high frequency (HF) ratio of the heart rate variability spectrum (LF/HF); and determining whether the LF/HF ratio of the heart rate variability spectrum is greater than a predetermined threshold value; wherein, if the LF/HF ratio of the heart rate variability spectrum is greater than the predetermined threshold value, the patient is particularly suitable for said treatment. For example, the predetermined threshold value of the LF/HF ratio of the heart rate variability spectrum may be about 1. The predetermined threshold value may also be calculated using the average low frequency (LF) to high frequency (HF) ratio of heart rate variability spectra from a healthy patient population and/or critical illness patient population. Alternatively, or in addition to, the predetermined threshold value may be calculated using the low frequency (LF) to high frequency (HF) ratio of a heart rate variability spectrum obtained from the user at one or more prior timepoints.

The present invention also provides a method of screening a patient suffering or recovering from a critical illness for treatment according to a method disclosed herein. The method comprises recording an electrocardiogram of a patient for a minimum period of 1 minute; analysing the power spectrum of heart rate variability; determining the low frequency (LF) value and the high frequency (HF) value of the power spectrum of heart rate variability; determining whether the LF value or the HF value of the power spectrum of heart rate variability is greater than a predetermined threshold value, wherein, if the LF value or the HF value of the power spectrum of heart rate variability is greater than the predetermined threshold value, the patient is particularly suitable for treatment according to the method disclosed herein.

Such screening methods of the invention may therefore also further comprise a step of administering a pharmaceutically active composition suitable for the treatment of the critical illness.

There is strong supporting evidence that acute injury and illness causes autonomic imbalance, which is characterized by sympathetic nervous system activation and parasympathetic (vagal) withdrawal. Autonomic dysfunction drives maladaptive remodelling and promotes deterioration in the heart and other organs, through multiple pathological mechanisms. Autonomic dysfunction is an independent predictor of death in ICU patients and reduced survival even if they survive to hospital discharge. The present invention therefore provides a method to treat such patients suffering from such critical illness conditions and aid their recovery thereafter.

### BRIEF DESCRIPTION OF THE DRAWINGS

Various embodiments of the invention are described by way of examples with references to the accompanying drawings in which:

FIG. 1 depicts sites on the left tragus and the right tragus of the human outer ear that receive sensory innervation. Electrical stimulation of these nerves transcutaneously or percutaneously modulates autonomic activity and can be used to treat critical illness and/or aid recovery from critical illness according to the present invention.

FIG. 2 depicts a description and circuit block drawing of a device for use according to the present invention.

FIG. 3 depicts a possible waveform of the electrical stimulation signal.

FIG. 4 depicts the results of tragal nerve stimulation (taVNS) on three patients in post-surgical recovery.

### DETAILED DESCRIPTION

The terms “subject”, “individual”, “user” and “patient” as used herein refer to humans, which do not denote a particular age or sex. In certain embodiments the individual subject may be a patient, a subject that is a candidate for, or awaiting medical or other treatment, such as the method of neuromodulation described herein. The term “about” as used herein means in quantitative terms plus or minus 10%.

Method and Device for Treating a Patient with a Critical Illness

The present invention employs a device and a specific method of neuromodulation to treat (and aid recovery from) critical illness via electrical stimulation of afferent (sensory) branches of cranial nerves innervating the tragus and projecting to the brainstem.

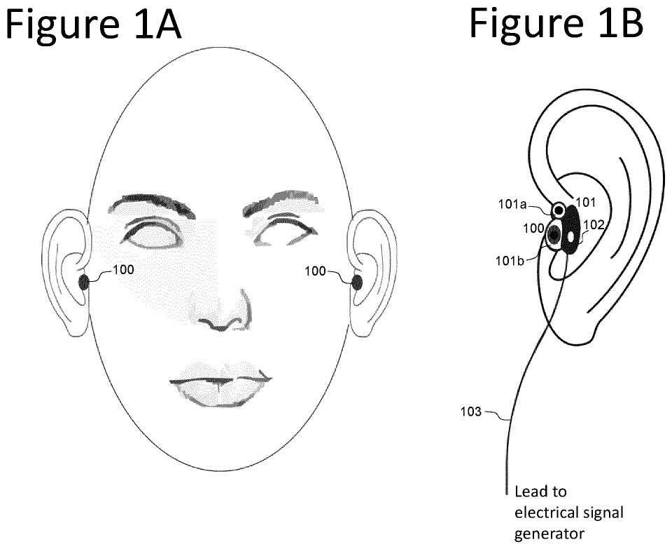

More specifically, the present invention aids recovery for a patient suffering from a critical illness by non-invasive neuromodulation, produced by a specific stimulation treatment programme involving delivery of electrical pulses with specific characteristics applied transcutaneously (to the skin) or percutaneously (using electrodes through the skin) to the inward and outward facing regions of the tragus on both ears (FIG. 1). At its broadest, the present invention facilitates recovery of patients with critical illness by stimulating cranial and spinal nerve fibers innervating the tragus region of the outer ear to produce autonomic neuromodulation. The autonomic remodulation produced helps to correct the autonomic dysfunction induced by the critical illness, thus redressing the autonomic balance toward a healthy state.

FIG. 1 illustrates the sites of electrical stimulation to activate cranial nerves.

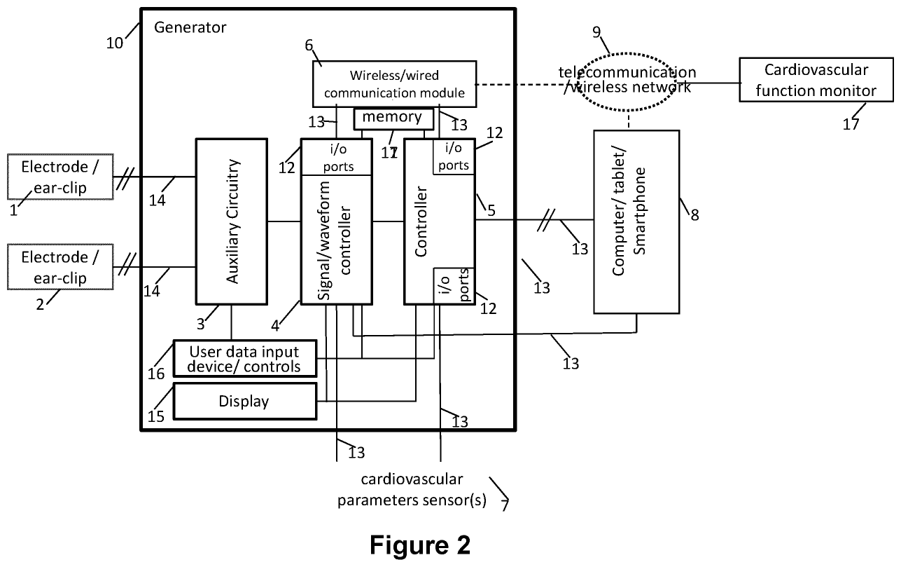

FIG. 2 depicts a device for treating a patient with a critical illness. The device comprises a generator configured to produce an electrical stimulation signal; a controller, connected to the generator and configured to determine the form of the electrical stimulation signal and an earpiece, connected to the generator and controller, the earpiece comprising an electrode. The earpiece is connected to the generator and controller via a lead. Alternatively, the earpiece may be connected to the generator and controller via a wireless or Bluetooth connection.

The controller is configured to produce the electrical stimulation signal and the pattern of stimulation based on a user input received at the controller. The controller can therefore adjust the electrical stimulation signal depending on the required treatment plan for the user. The controller may be connected to a communication module to deliver information to the controller from an external source. Alternatively, the controller can be controlled by the user of the device directly. The user input includes at least one of the pulse duration, waveform, pulse frequency, pulse pattern and current amplitude of the electrical stimulation signal. The user input may also include information on the duration of usage of the device and interval period between using the device for subsequent rounds of treatment. For example, informing user that the device is to be used for a period of between 5 min and 2 hours each day for a minimum of 3 consecutive days.

FIG. 1A depicts the schematic depiction of a human head and depicts the tragus 100 on each ear. The regions of particular interest for the present invention are the left tragus and the right tragus 100. FIG. 1B depicts placements of stimulating electrodes to activate sensory nerves innervating the tragus region of the outer ear. The same stimulating electrodes can be applied to each of the left tragus and the right tragus, known as bilateral stimulation. Improved results are detected when using bilateral stimulation compared to using stimulation of just one tragus. Two electrodes may be placed on each tragus, one facing inwards and one facing outwards. The device enables both the left and the right tragi to be electrically stimulated for the purpose of autonomic neuromodulation. The stimulation signal generated by the device may be applied to each of the left or right tragus on their own or both simultaneously.

The controller transmits the electrical stimulation signal to the electrode(s) and the electrode(s) is are configured to be placed in contact with and provide the electrical stimulation signal to the skin of a tragus of the user (i.e. such as across the tragus).

The electrical stimulation signal applied to the left and right tragi is substantially the same electrical stimulation signal (i.e. has substantially the same waveform). Alternatively, the electrical stimulation signal applied to the left tragus is different to the electrical stimulation signal applied to the right tragus (i.e. has a different waveform). The electrical stimulation signal applied to the left and right tragi is applied simultaneously or sequentially to each of the left and right tragi. ‘Simultaneously’ means that the electrical stimulation signal is applied to the left and right tragus at substantially the same time. ‘Sequentially’ means that the electrical stimulation signal is first applied to one of the left or right tragus and is then subsequently applied to the opposite tragus. This action can be repeated several times to continuously apply the electrical stimulation signal to each of the left and right tragus in turn (for example, in 5 second intervals). Alternatively, the electrical stimulation signal can be applied to the left tragus at a different time to the electrical stimulation signal applied to the right tragus. ‘Different’ means that the electrical stimulation signal may be applied to only one of the left or right tragus and not both at the same time. The device further includes a securing means to secure the electrode to the tragus and hold it in place. This is used to secure the electrode to the tragus for an extended period of time such that the treatment can be continuously delivered to the user. The securing means is configured to secure the earpiece and the electrodes in place over the skin of the tragus. The securing means may include a clip or the earpiece itself may take the form of a clip. For example, in some embodiments, the clip may be configured to secure the earpiece electrodes in place by gripping a user's tragus, with a first gripping portion and a second gripping portion on respective sides of the tragus. Where this is the case, the stimulating electrode may be located on the first gripping portion, and a reference electrode may be located on the second gripping portion. One or both of the first gripping portion and the second gripping portion may extend into the ear canal. The physiological sensor may also be located on the clip and is for example, also located on the first gripping portion or the second gripping portion. Alternatively, the physiological sensor may be present on part of the device which is not the clip.

In some embodiments, the clip is shaped to provide an ergonomic fit on the tragus. This is advantageous for delivery of electrical pulses and for precise monitoring of physiological parameters such as heart rate and blood pressure, while minimizing motion-related artefacts in the sensor signal (or physiological signal such as heart rate).

FIG. 1B depicts stimulating electrodes in place on the tragus of a user. Specifically, a stimulating electrode and a reference electrode are embedded in a tragus clip 101, which has two lobes 101a and 101b which are biased to provide a gripping force on the tragus 100 of the user of the device. Lobes 101a and 101b are positioned on either side of the tragus and are biased against each other to hold the tragus clip in place. The lobes 101a and 101b are positioned against the skin of the tragus. Lobe 101a includes a stimulating electrode, and the opposite lobe 101b includes a reference electrode, which are arranged to provide an electrical stimulation signal to the tragus. The lobe 101a includes a reference electrode, and the opposite lobe 101b includes a stimulating electrode, which are arranged to provide an electrical stimulation signal to the tragus. The earpiece 101 may also include a physiological monitor 102 which is configured to record the heart rate, blood pressure, and/or temperature and store the value in a memory portion of the device. Earpiece 101 is connected to a device that generates the electrical signal by a lead 103.

The clip has a first gripping portion and a second gripping portion which may correspond to two lobes 101a and 101b which are biased to provide a gripping force on the tragus 100 of the user of the device. The stimulating electrode is located on the first gripping portion and the reference electrode is located on the second gripping portion.

The device has a first and second electrode (not shown), the first electrode is configured to be placed in contact with the left tragus of the user and the second electrode is configured to be placed in contact with the right tragus of the user. Additionally, the device has a first earpiece and a second earpiece, and the first earpiece comprises the first electrode and the second earpiece comprises the second electrode. Therefore, the configuration shown in FIG. 1B can be applied to each ear. The device may be applied to just one ear or alternatively to both ears at the same time. The device is configured to be placed in contact with an outward facing surface and an inward facing surface of the tragus. The reference electrode is in contact with a first surface of the tragus and the stimulating electrode is in contact with a second surface of the tragus. The first surface of the tragus may, for example, be facing inwards (i.e. towards the head of the user) and the second surface of the electrode is therefore facing outwards (i.e. away from the head of the user).

The electrode can be placed in contact with the skin via either a transcutaneous or a percutaneous contact. Where the contact is transcutaneous, this means that the electrode is placed on but not piercing the skin. Where the contact is percutaneous this means that the electrode may have needles or electrodes that directly pierce the skin. The needle or electrode may pierce the skin to deliver the electrical stimulation signal to the user.

In some embodiments the first and the second earpiece are substantially identical. In some embodiments the first earpiece is shaped to fit on the left tragus and the second earpiece is shaped to fit on the right tragus. In some embodiments the left and the right earpieces are substantially mirror images of each other. In some embodiments an earpiece is shaped to conform to a portion of the tragus or another part of the ear, such that the earpiece fits preferentially to the tragus such that the stimulating electrode is in contact with the outer side of the tragus and the reference electrode is in contact with the inner side of the tragus. In some embodiments an earpiece is shaped to conform to a portion of the tragus or another part of the ear, such that the earpiece fits preferentially to the tragus such that the stimulating electrode is in contact with the inner side of the tragus and the reference electrode is in contact with the outer side of the tragus.

The device may comprise a stimulating electrode, a counter electrode and a reference electrode, and may be configured to measure one or both of: the voltage between the stimulating electrode and the reference electrode, and the voltage between the counter electrode and a reference electrode. In this way, the potential of the stimulating and/or the counter electrodes may be measured without error arising from voltage drop across the electrode to skin interface.

The application of electrical current pulses to the skin of the tragus is done across the tragus, with a stimulating and a reference an electrodes placed on either side of the tragus. In this way a net current flow is provided across the tragus. In some embodiments the net conventional current flow is positive. In other embodiments the net conventional current flow is negative.

In some embodiments the electrical stimulation signal comprises a cyclically repeating multiphasic pulse waveform in which the amplitude and/or duration of the phases of the pulse at which the signal at the first stimulating electrode is positive with respect to the signal at the reference second electrode is/are greater than the amplitude and/or duration of the phases of the pulse at which the signal at the first electrode is negative with respect to the signal at the second electrode. In this way, a net conventional current flow is provided over the duration of the pulse that is positive from the first electrode to the second electrode.

In some embodiments, the method comprises applying the electrical stimulation signal to the tragus of the user such that the current flow between the stimulating electrode and the reference electrode first and the second electrodes is primarily through the tissue of the tragus, and negligibly through tissue that does not form part of the tragus.

In some embodiments the method comprises applying the electrical stimulation signal to the tragus of the user such that the current flow between the stimulating electrode and the reference electrode first and the second electrodes is primarily or exclusively between the outer and the inner surfaces of the tragus, through the tissue of the tragus.

In some embodiments the electrical stimulation signal comprises a cyclically repeating series of pulses; and the electrical stimulation signal is selected such that during each cycle there is a net conventional current flow from the stimulating electrode to the first electrode to the second electrode.

In some embodiments the electrical stimulation signal is selected such that over the course of a series of cyclically repeating pulses, there is a net conventional current flow from the stimulating electrode to the first electrode to the second electrode.

The stimulating electrode is configured to deliver the electrical stimulation signal to the user for treatment purposes. On the other hand, the reference electrode is used to take alternative measurements via a sensor. For example, the reference electrode may take sensor measurements of features such as temperature and physiological parameters. These may be used to determine the user's compliance with the device and method, for example, which may have been prescribed by a doctor or medical member of staff. In particular, the reference electrode may take temperature measurements to determine whether the device is in contact with human skin. Alternatively, the reference electrode could also sense the pulse of the user.

The device measures the physiological parameters and temperature parameters and stores these within a memory portion of the device. The measurements of these parameters are stored alongside date and time stamp information, such that a record can be kept.

Optionally, the device may include electronic circuitry and cardiovascular function sensors to measure and monitor the voltage, current and phase relationship of the stimulating electrical signal. Measurements of current, voltage and phase relationship of the electrical stimulation signal are stored in a memory portion of the device and are used to determine the electrical impedance of the tragus. Measurements of electrical impedance are used to sense that the electrodes are connected to a human and/or monitoring/measuring the cardiovascular function. The measurements of electrical impedance are also stored in the memory portion of the device.

The information stored in the memory portion can be used to determine the electrical stimulation signal, for example, by adjusting the form of the signal depending on the information retained in the memory. The memory portion of the device can be accessed by a third party.

The device may further comprise a communication module connected to the controller and the memory portion of the device. The communication module is configured to send information from the device to an external computer system and to receive information from the external computer system. This information can be used to inform the patient's treatment plan and determine compliance with the prescribed treatment plan. The information received from the external computer system is used by the controller to determine the form of the electrical stimulation signal. Therefore, the electrical stimulation signal may be remotely controlled by a third party. Information shared via the communication module may include the physiological and temperature measurements taken during use of the device. The communication module and device together form a system for modulating blood flow to the brain of a user.

To achieve a therapeutic effect, electrical stimulation of the sensory nerves innervating the tragus requires application of current pulses with the following specific parameters: frequency 1-100 Hz, amplitude 0.1-8 mA, pulse width 10-500 microseconds using a square shape pulse. Transcutaneous application of electrical current pulses at frequencies between 1-100 Hz, amplitudes between 0.1-8 mA, and pulse widths between 10-500 microseconds triggers reliable action potential firing in the subcutaneous nerve fibers innervating the tragus, resulting in autonomic neuromodulation. Application of current pulses at frequencies lower or higher than the 1-100 Hz range, amplitudes smaller or higher than 0.1-8 mA range, and pulse widths shorter or longer than 10-500 microseconds range is without the therapeutic effect. In an example, the electrical stimulation of the cranial nerves innervating the tragus can be applied using pulses with the following specific parameters: frequency 1-30 Hz, amplitude 0.1-8 mA, pulse width 10-200 microseconds, using a square shape pulse. This example range is shown to produce the greatest therapeutic effect.

Neuromodulation by electrical stimulation of the tragus may also include a course of treatment involving several sessions of stimulation in accord with the following stimulation treatment programme: stimulation is applied daily to the left and right tragus simultaneously (i.e. bilaterally) for a period of between 5 min and 2 hours each day for a minimum of 3 consecutive days (initial course of treatment). Then the stimulation may be applied once a week (every 7 days) to the left and right tragus simultaneously for a period of up to 2 hours each session during the course of treatment (subsequent course of treatment). The stimulation can alternatively be applied several times a day. For example, the device can be applied to the user throughout several time periods in the day. The total sum of all daily usage of the device may add up to a period of between 5 min and 2 hours each day.

If after the initial course of treatment the recovery of the patient from the critical illness is not apparent (e.g. partial recovery), further subsequent courses of treatment may be administered to achieve and maintain the therapeutic effect. This cycle of treatments may be applied on a regular basis for as long as the critical illness condition, and/or critical illness-induced functional impairment, exists.

It is possible to screen patients to identify those patients who are particularly suitable for treatment. To achieve this, a patient's electrocardiogram is recorded for a minimum period of 1 min and the power spectrum of heart rate variability is analysed to determine the low frequency (LF) to high frequency (HF) ratio (LF/HF) of the heart rate variability spectrum. The method of neuromodulation via stimulation of the cranial sensory innervation of the tragus is expected to aid functional recovery after critical illness in patients (for example, by reducing blood pressure in critical illness patients and/or improving cardiac function in patients with heart failure) whose LF/HF ratio of their measured heart rate variability spectrum is larger than 1 (one).

The device for treatment of patients with a critical illness according to a method of the present invention may include an earpiece having stimulating electrode(s) configured to provide an electrical stimulation signal to the tragus, a generator connected to the stimulating electrode for generating the electrical stimulation signal, a controller connected to the generator, for determining both the form of the electrical stimulation signal and the pattern of stimulation. Optionally, the device may include electronic circuitry and cardiovascular function sensors to measure and monitor the voltage and current of the stimulating electrical signal as well as cardiovascular physiological signals (e.g. ECG, blood pressure); a micro-controller or computer, memory, user input keypads and peripheral devices, physiological sensors, display element, and associated circuitry to input, control and record data associated with the device, a wireless and/or wired communications system for interfacing with external devices, and a computer, tablet and/or smartphone external to the device to enable the device to be programmed and to enable transfer of data and information to and from the device.

FIG. 2 depicts a circuit block for a device according to the present invention. The generator may comprise a signal/waveform generator 4, a controller 5, memory 11, auxiliary circuitry 3, user data input/control device(s) 16 (such as keypads, dials, actuator/switches), display device 15, communication modules (wireless and/or wired communication), 6. The illustrated generator 10 further includes a transceiver or communication module and other input/output circuit(s) (i/o ports) 12. The i/o ports allows the generator device to communicate with other devices 8, and thus can be used to program the generator device and/or upload historical generator data recorded over a period of time, for example. The i/o ports 12 may include switch (such as mechanical, electrical, electronic and magnetic) providing a means for initiating a programmed stimulation algorithm which may be triggered by a physician, healthcare professional or patient.

The generator 10 delivers electrical stimulation (stimulation algorithm) using a defined schedule to produce autonomic neuromodulation and aid functional recovery after critical illness (for example, by modulating brain blood flow and chronically lowering blood pressure, and/or reducing AF burden and improving cardiac function). According to various embodiments, the device further includes at least one port 12 to connect to at least one lead 13 which may be part of the controller 4 and/or the micro-controller 5 (FIG. 2). Thus, for example, the lead(s) 13 is/are capable of detaching from the device 10, and other leads are capable of being used with the device. As is described above, the generator is for determination of the electrical stimulation signal. More specifically, the generator may be for the determination of time-course parameters related to the stimulation algorithm, such as pulse width, pulse frequency, waveform and waveform pattern here on referred to as the waveform. Examples of the waveform pattern include, but not restricted to, sinusoidal, square, triangular, and “white noise” signals. The controller for example, produces the electrical parameters of the stimulation algorithm (current and/or voltage amplitude, frequency, burst-frequency, waveform and duration) of stimulation algorithm hereon referred to as the waveform parameters based on the signal determined by and received from the generator. The generator may also determine the waveform parameters based on the sensor signal. This determination may take place in two ways: user-controlled utilising the display 15 and/or user data input/control device(s) 16, or automatically from pre-programmed, computer-readable instructions determined by a micro-controller.

The controller 4, micro-controller 5, memory 11, auxiliary circuitry 3, user data input/control device(s) 16 (such as keypads, dials switches), display device 15, communication modules (wireless and/or wired communication), 6 may be located within the same component, which may be a portable battery-operated electronic device. The portable electronic device is for example able to run applications or apps, and is for example a laptop computer, a tablet or a smartphone 8. Alternatively, the generator may be in the form of a portable electronic device, and the generator may be a separate component.

The controller 4 may further be connected to the Auxiliary Circuit 3 to provide a stimulation algorithm to at least one electrode 1 and for example two electrodes 1 & 2 to stimulate at least one and for example both tragus(s) of a human ear(s) when an appropriate signal is provided to the electrode or electrodes.

A stimulation algorithm is provided using a single lead and a single electrode on the lead. However, multiple leads and multiple electrodes on the leads can be used. The electrode and/or 1 & 2 may be of a wearable device is for example connected to the generator device via an electric cable 14, or via a wireless connection 14 such as Bluetooth. Some embodiments where more than one electrode is used to stimulate the patient, the same or different waveforms may be applied to two or more electrodes. The two different waveforms may vary in pattern and/or waveform parameters.

The generator may be an open loop or closed loop system and controlled by computer-readable instructions. In the closed loop embodiment the stimulation algorithm may be adapted in response to signals from the cardiovascular parameters sensor(s) 7 which may include one or plurality of sensors such as a blood pressure sensor, temperature sensor, pulse oximetry, electrocardiogram sensor, heart rate sensor, temperature and tissue impedance sensor designed to sense a parameter indicative that the electrodes are connected to a human and/or monitoring/measuring the cardiovascular function where the stimulation algorithm is adapted to produce autonomic neuromodulation using the sensed parameter. Thus, the closed loop system is capable of employing information from the cardiovascular sensors as a feedback mechanism and/or programme to reduce and/or increasing the stimulation intensity, alter and/or change the waveform and/or waveform parameters as appropriate to maintaining some measured physiological parameters within an upper and lower boundary during the stimulation. In the open loop embodiment, the stimulation algorithm is adapted to lower heart rate and/or adjusting the waveform parameters using an external device 8. Additionally, in various embodiments, the generator is adapted to set parameters of the stimulation signal and, in some embodiments, vary parameters of the stimulation algorithm to adjust the intensity of the stimulation using either the user data input device 16 and/or an external computer 8.

The memory 17 (or memory portion) includes computer-readable instructions that are capable of being operated on by the controller and/or micro-controller to perform functions of the device. Thus, in various embodiments, the generator is adapted to operate on the instructions to provide programmed stimulation algorithm such as anti-hypertensive and atrial fibrillation improvement therapies. Additionally, in various embodiments, the generator is adapted to set parameters of the stimulation signal and, in some embodiments, vary parameters of the stimulation signal to adjust the intensity of the stimulation algorithm, such as is generally illustrated by the stimulation intensity as illustrated in FIG. 2.

The micro-controller and memory devices may include pre-programmed computer-readable instructions to provide controlled electronic access to the generator, implement security, password and encryption feature to limit access to the device and stored data and store information such as the user and maintenance instruction, device specific data required by legal and/or regulatory statutes.

According to various embodiments, a single or plurality of physiological parameters of a patient may be measured by means of the cardiovascular sensors and recorded by the generator. Further, several such measurements may be made at different times during a single stimulation algorithm period or at different times over more than one stimulation event to establish the value and/or range of values for any particular physiological parameters. In one such embodiment, the magnitude of the voltage and/or current of the stimulation algorithm and their phase relationship may be used to determine the electrical impedance of the patient's skin and be recorded by the generator. Thus, such physiological parameters value or range of values recorded during the stimulation algorithm period in conjunction with the usage of the device (date, time, waveform and waveform parameters) may be recorded and combined to construct a data-set (individual usage data), indicative of the patient usage of the device thus providing and recording information on the use of the generator by a subject. This information may further be reported to the external device such that the information may be used monitor a patient condition, to demonstrate and/or provide evidence of a patient's compliance with the stimulation/treatment programme as prescribed by a physician, healthcare professional or healthcare agreement, contract or health insurance agreement with a third party. Further, the individual usage dataset may be accessed remotely by means of the communication module to enable remote monitoring of patients, validating a patient's compliance with healthcare plan and prescribed treatment, adapting initial and subsequent course of stimulation and the stimulation algorithm to change prescribed treatment.

According to various embodiments, the device may communicate with an external computer, tablet or smartphone whereby by the computer, tablet or smartphone is further able to communicate with a cardiovascular function monitor such as a blood pressure monitor and/or heart-rate monitor and/or ECG monitor to record individual usage data and data from the cardiovascular function monitor(s). A physician or other healthcare professional may assess the performance of the patient's cardiovascular system and advise the patient in person, or remotely (via the internet or other telecommunication/computer network) 18, on the initial course of treatment and/or subsequent course of treatment.

The device may include a plurality of stimulation electrodes on one earpiece, and may include a reference electrode associated with each stimulation electrode, or a single reference electrode associated with the plurality of stimulation electrodes on a given earpiece. Specifically, there may be a plurality of stimulating electrodes configured to provide an electrical stimulation signal to the tragus.

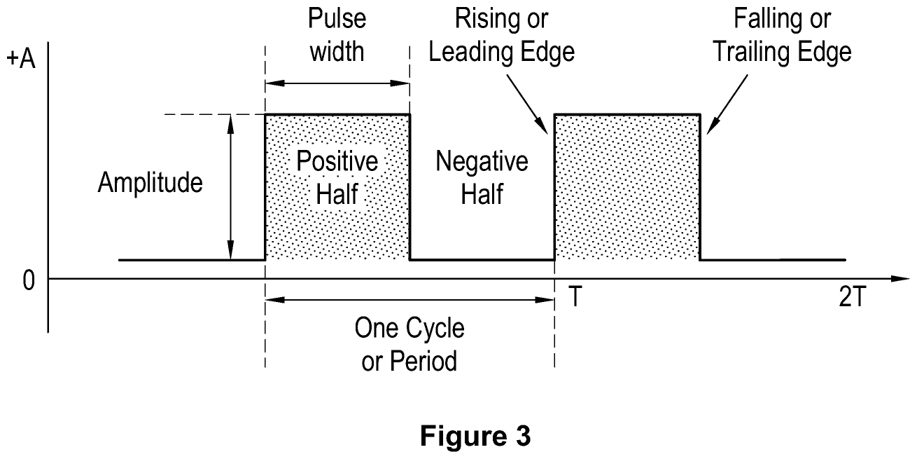

As shown on FIG. 3, the electrical stimulation signal has an amplitude, a pulse width and a frequency. The amplitude is the magnitude or intensity of the signal waveform measured in volts or amps (measured by the difference between the highest and lowest part of the waveform). The frequency is the number of times the waveform repeats itself within a one second time period, measured in Hz. The pulse width is the length of time in seconds that the waveform takes to repeat itself from start to finish. A square wave is illustrated in FIG. 3, however, the waveform can take any shape including a sinusoidal, square, triangular or ‘white noise’ waveform.

The generated waveform can be a symmetrical monophasic waveform, or a symmetrical biphasic waveform, or a symmetrical triphasic waveform. The generated waveform can be an asymmetrical monophasic waveform, or an asymmetrical biphasic waveform, or an asymmetrical triphasic waveform. It is feasible for the waveform to take any of these shapes.

### Optimised Treatment Programmes

To achieve a therapeutic effect, electrical stimulation of the sensory innervation of the tragus is for example applied using an electrical stimulation signal with the following parameters: frequency 1-30 Hz, amplitude 0.1-4 mA, pulse width 10-200 microseconds. It is also desirable to use a square shape pulse. The therapeutic effect can be achieved by using a frequency of no less than 1 Hz and no more than 100 Hz. Furthermore, the therapeutic effect can also be observed by using a frequency of no less than 3 Hz and no more than 50 Hz The therapeutic effect can be achieved by using a pulse width of no less than 10 microseconds, and no more than 500 microseconds and amplitude of no less than 0.1 mA, and no more than 8 mA. The therapeutic effect can also be achieved by using a pulse width of no less than 100 microseconds, and no more than 500 microseconds. It is possible to optimise just one of the parameters, such as frequency, amplitude or pulse width, and it is not required to optimise all to produce the therapeutic effect. For example, the electrical stimulation signal may have a pulse that repeats with a frequency of 1 Hz to 100 Hz or each pulse having duration of 10 microseconds to 500 microseconds or an amplitude of 0.1 mA to 8 mA.

For completeness we submit that in some embodiments, the electrical stimulation signal comprises a series of electrical pulses, each pulse repeating with a frequency in the range about 3 Hz to about 50 Hz and each pulse having a duration of about 100 microseconds to about 500 microseconds and an amplitude of about 0.1 mA to about 8 mA.

In some embodiments the frequency is in the range from about 1 Hz to about 100 Hz, such as about 1 Hz to 10 Hz, 10 Hz to 20 Hz, 20 Hz to 30 Hz, 30 Hz to 40 Hz, 40 Hz to 50 Hz, 50 Hz to 60 Hz, 60 Hz to 70 Hz, 70 Hz to 80 Hz, 80 Hz to 90 Hz, or 90 Hz to about 100 Hz.

In some embodiments the frequency is in the range 3 Hz to 20 Hz, 5 Hz to 30 Hz, 10 Hz to 50 Hz, 15 Hz to 60 Hz, 20 Hz to 75 Hz, 25 Hz to 80 Hz, 30 Hz to 100 Hz.

In some embodiments the frequency is in the range 3 Hz to 50 Hz. In some embodiments the frequency is in the range about 3 Hz to about 35 Hz.

In some embodiments the pulse has a duration in the range about 10 microseconds to about 500 microseconds, such as about 10 microseconds to 100 microseconds, 20 microseconds to 200 microseconds, 30 microseconds to 300 microseconds, 40 microseconds to 400 microseconds, 50 microseconds to about 500 microseconds.

In some embodiments the pulse has a duration in the range 100 microseconds to 200 microseconds, 200 microseconds to 300 microseconds, 300 microseconds to 400 microseconds, 400 microseconds to 500 microseconds.

In some embodiments the pulse has a duration in the range 50 microseconds to 200 microseconds, 100 microseconds to 250 microseconds, 200 microseconds to 500 microseconds.

In some embodiments the pulse has a duration in the range 100 microseconds to 500 microseconds. In some embodiments the pulse has a duration in the range about 100 microseconds to about 300 microseconds.

In some embodiments the amplitude is in the range about 0.1 mA to about 10 mA, such as about 0.1 mA to about 2 mA, about 0.2 mA to about 5 mA or about 0.5 mA to about 10 mA.

In some embodiments the amplitude is in the range 0.1 mA to 1 mA, 0.2 mA to 2 mA, 0.3 mA to 3 mA, 0.4 mA to 4 mA, 0.5 mA to 5 mA, 0.6 mA to 6 mA, 0.7 mA to 7 mA, 0.8 mA to 8 mA, 0.9 mA to 9 mA or 1.0 mA to 10 mA.

In some embodiments the amplitude is in the range 0.1 mA to 5 mA, 0.5 mA to 8 mA or 1 mA to 10 mA.

In some embodiments the amplitude is in the range about 0.5 mA to about 5 mA.

In some embodiments the amplitude is in the range about 0.1 mA to about 20 mA.

Neuromodulation by electrical stimulation of the tragus may require a course of treatment involving several sessions of stimulation in accord with the following stimulation treatment programme: stimulation is applied daily to the left and right tragus simultaneously (i.e. bilaterally) for a period of between 5 min and 2 hours each day for a minimum of 3 consecutive days (initial course of treatment). The therapeutic effect can be optimised by applying the method electrical stimulation treatment to the user a minimum of 5 minutes and a maximum of 2 hours per day. The electrical stimulation is applied to the user separated by intervals of at least one day. Then the stimulation may be applied once a week (every 7 days) to the left and right tragus simultaneously for a period of up to 2 hours each session during the course of treatment (subsequent course of treatment).

Additionally, further treatment plans shown to be effective involve applying the electrical stimulation to the user using different periods. During a first period the method is applied to the user for between 5 minutes and 2 hours each day. The first period is typically a minimum of 3 consecutive days, although can be applied for longer depending on the needs of the patient to achieve a therapeutic effect. During a second period the method is stopped for at least 2 days. During a third period the method is applied to the user for between 5 minutes and 2 hours each day.

The electrical stimulation may be applied to the user in combination with any medications administered according to the clinical guidelines for modulation of pharmacological effect.

Furthermore, it is possible to adjust the treatment programme of the user in response to measurements of physiological parameters. The treatment can involve continuous measurement of the patient's physiological parameters. For example, the blood pressure can be measured compared to a predetermined threshold value (for example, a blood pressure considered to be healthy). The threshold value may be set by the user or by a third-party controller. The third-party controller may communicate with the device via the communication module. Firstly, measurements of the user's blood pressure are taken and recorded in the memory portion of the device. Then the controller determines whether the user's blood pressure is greater than a predetermined threshold value; and if the user's blood pressure is greater than the predetermined threshold value, the generator is instructed to produce the electrical stimulation signal. The predetermined threshold value is calculated to by determining an improvement based on a patient's pre-simulation physiological parameters. This is calculated by measuring the patient's physiological parameters (e.g. LF, HF, or LF/HF) pre-stimulation with the device and measuring the patient's physiological parameters post-stimulation. If there is a change in the patient's physiological parameters that is greater than 20% then it is determined that the predetermined threshold value has been met.

### Example 1: Application of Tragal Nerve Stimulation (taVNS) on Three Patients in Post-Surgical Recovery

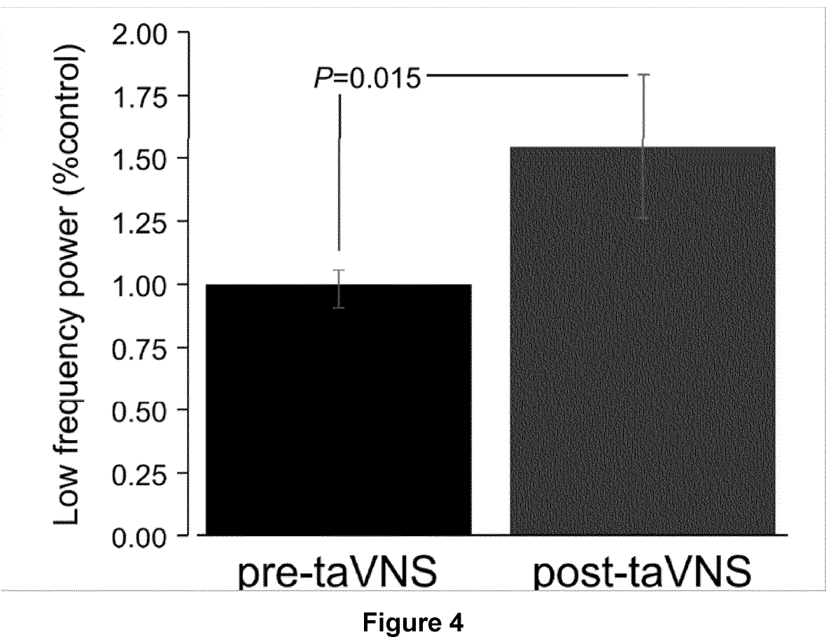

In 3 patients recovering after trauma post-surgery, taVNS increased a component of autonomic function that reflects baroreflex sensitivity which is critical for optimal blood pressure control, termed LF [see: Clin Won Res 2011 June; 21(3): 33-141]. The results shown confirm that taVNS restores normal autonomic function (see FIG. 4). tVNS was applied for 30 minutes after a control period of heart rate recording, using an ECG Holter monitor. Autonomic measures before and after the 30-minute period were compared. The procedure has not been used previously in patients. Enhancing baroreflex control reduces fluctuations in blood pressure, where high and low blood pressures are associated with further organ injury. The results of the treatments are shown in FIG. 4.

Features of the above aspects can be combined in any suitable manner. It will be understood that the above description is of specific embodiments by way of aspect only and that many modifications and alterations will be within the skilled person's reach and are intended to be covered by the scope of the appendant claims.

1. A method for the treatment of a patient suffering or recovering from a critical illness using a device comprising a generator configured to produce an electrical stimulation signal and a controller, connected to the generator and configured to determine the form of the electrical stimulation signal, the method comprising:
(i) producing an electrical stimulation signal from the generator, and determining the form of the electrical stimulation signal using the controller connected to the generator; and
(ii) transmitting the electrical stimulation signal to an electrode in contact with a tragus of the patient.

2. The method for the treatment of a patient suffering or recovering from a critical illness according to claim 1, wherein the electrical stimulation comprises a series of electrical pulses.

3. The method for the treatment of a patient suffering or recovering from a critical illness according to claim 2, wherein each electrical pulse repeats with a frequency of 1 Hz to 100 Hz.

4. The method for the treatment of a patient suffering or recovering from a critical illness according to claim 2, wherein each pulse has a duration of 10 microseconds to 500 microseconds.

5. The method for the treatment of a patient suffering or recovering from a critical illness according to claim 2, wherein each pulse has an amplitude of 0.1 mA to 8 mA.

6. The method for the treatment of a patient suffering or recovering from a critical illness according to claim 1, wherein the device is applied to the patient at least once a day.

7. The method for the treatment of a patient suffering or recovering from a critical illness according to claim 1, wherein the device is applied to the user for a minimum of 5 minutes and a maximum of 2 hours per day.

8. The method for the treatment of a patient suffering or recovering from a critical illness according to claim 7, wherein the device is applied to the user for a minimum of 15 minutes and a maximum of 2 hours per day.

9. The method for the treatment of a patient suffering or recovering from a critical illness according to claim 1, wherein
during a first period the method is applied to the user for between 5 minutes and 2 hours each day, the first period comprising a minimum of 3 consecutive days,
during a second period the method is stopped for at least 2 days, and
during a third period the method is applied to the user for between 5 minutes and 2 hours each day.

10. The method for the treatment of a patient suffering or recovering from a critical illness according to claim 9, wherein the method is applied to the user for between 15 minutes and 2 hours each day.

11. The method for the treatment of a patient suffering or recovering from a critical illness according to claim 1, wherein the critical illness is a disease or condition including at least one of COVID-19 infection, post-surgical recovery, trauma, post-traumatic stress disorder (PTSD), sleep apnoea, infection (viral, bacterial and/or fungal infection, including sepsis), organ failure, e.g. failure of the heart, lung, brain, kidneys, bone marrow and/or other major organs, cancer, heart attack, organ transplant (e.g. heart, liver, kidney, lung, bone marrow transplant), coronary bypass surgery and angioplasty.

12. The method for the treatment of a patient suffering or recovering from a critical illness according to claim 1, wherein the method further comprises a step of administering to the patient a pharmaceutically active composition for treatment of the critical illness.

13. The method according to claim 1, further comprising:
screening a patient suffering or recovering from a critical illness for treatment including:
(i) recording an electrocardiogram of a patient for a minimum period of 1 minute; (ii) analysing the power spectrum of heart rate variability; determining the low frequency (LF) to high frequency (HF) ratio of the power spectrum of heart rate variability (LF/HF);
(iii) determining whether the LF/HF ratio of the power spectrum of heart rate variability is greater than a predetermined threshold value,
wherein, if the value of the LF/HF ratio of the power spectrum of heart rate variability is greater than the predetermined threshold value, the patient is particularly suitable for treatment according to the method of claim 1.

14. The method according to claim 13, wherein the threshold value is about 1.

15. The method according to claim 1, further comprising:
screening a patient suffering or recovering from a critical illness for treatment including:
(i) recording an electrocardiogram of a patient for a minimum period of 1 minute; (ii) analysing the power spectrum of heart rate variability; determining the low frequency (LF) value and the high frequency (HF) value of the power spectrum of heart rate variability;
(iii) determining whether the LF value or the HF value of the power spectrum of heart rate variability is greater than a predetermined threshold value,
wherein, if the LF value or the HF value of the power spectrum of heart rate variability is greater than the predetermined threshold value, the patient is particularly suitable for treatment according to the method of claim 1.
